(19) United States
(12) Patent Application Publication
(10) Pub. No.: US 2010/0172883 A1
(43) Pub. Date: Jul. 8, 2010

(54) METHODS OF GENERATING CARDIOMYOCYTES

(76) Inventors: Benoit Gaëtan Bruneau, Oakland, CA (US); Junk K. Takeuchi, Yokohama (JP)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/523,307

(22) PCT Filed: Jan. 18, 2008
(86) PCT No.: PCT/US08/00695

§ 371 Date: Mar. 8, 2010

Related U.S. Application Data
(60) Provisional application No. 60/881,795, filed on Jan. 19, 2007.

(51) Int. Cl. A61K 35/34; A01N 1/00; C12N 15/09; C12N 5/02; C12N 5/0789

(52) U.S. Cl. 424/93.7; 435/1.1; 435/455

(57) ABSTRACT

The present invention provides methods of generating cardiomyocytes from a cell other than a cardiomyocyte, the methods generally involving contacting the cell with an agent that increases the level and/or activity of a protein that links a transcription factor to a chromatin remodeling complex. The present invention provides a population of cardiomyocytes generated using a subject method; and treatment methods involving introducing the cardiomyocyte population in or around diseased myocardial tissue.

### CROSS-REFERENCE

This application claims the benefit of U.S. Provisional Patent Application No. 60/881,795 filed Jan. 19, 2007 which application is incorporated herein by reference in its entirety.

### BACKGROUND

Cardiac tissue death can lead to other heart dysfunctions. If the pumping ability of the heart is reduced, then the heart may remodel to compensate; this remodeling can lead to a degenerative state known as heart failure. Heart failure can also be precipitated by other factors, including valvular heart disease and cardiomyopathy. In certain cases, heart transplantation must be used to repair an ailing heart. Unlike skeletal muscle, which regenerates from reserve myoblasts called satellite cells, the mammalian heart has a very limited regenerative capacity and, hence, heals by scar formation.

During organogenesis, the integration of transcriptional inputs coordinates the de novo deployment of an entire cell-specific gene expression program, so that lineage-committed precursor cells differentiate into a completely new cell type. This is apparent in the developing heart, which undergoes morphogenesis concomitant with differentiation of committed precursor cells into specialized cardiac myocytes. The transcriptional regulation of heart differentiation has been extensively studied, and although several transcription factors are important for activation of cardiac genes, no single transcription factor or combination of factors has been shown to activate the cardiac gene program de novo in mammalian cells. This is in contrast to other muscle cell types, which can be programmed by a single transcription factor, such as MyoD for skeletal muscle and myocardin for smooth muscle. Thus, the recalcitrance of non-cardiac cells to express cardiac genes and the transcriptional basis of cardiac differentiation are not fully understood.

There is a need in the art for methods of inducing cells to undergo cardiomyogenesis.

### Literature

Lickert et al. (2004) Nature 432:107; US 2006/0040389; Olson (2006) Science 313:1922-1927; Srivastava (2006) Cell 126:1037-1048.

### SUMMARY OF THE INVENTION

The present invention provides methods of generating cardiomyocytes from a cell other than a cardiomyocyte, the methods generally involving contacting the cell with an agent that increases the level and/or activity of a protein that links a transcription factor to a chromatin remodeling complex. The present invention provides a population of cardiomyocytes generated using a subject method; and treatment methods involving introducing the cardiomyocyte population in or around diseased myocardial tissue.

### BRIEF DESCRIPTION OF THE DRAWINGS

FIG. 1. Schematic for Baf60c-mediated activation of cardiac genes in 10T1/2 fibroblast cells.

FIG. 2 is a graph of percent embryos with ectopic Actc-positive foci (grey bars) and beating tissue (black bars).

FIG. 3 is a graph showing percent Actc-positive and beating embryos transfected with various transcription factors, alone or in combination with other transcription factors or with Baf60c.

FIG. 4 depicts quantitation of Actc induction by Gata4/Baf60a, Gata4/Baf60b, Gata4/Baf60c, or Gata1/Baf60c.

FIGS. 5A and 5B depict Baf60c amino acid and nucleotide sequences, respectively.

FIGS. 6A and 6B depict Gata4 amino acid and nucleotide sequences, respectively.

FIGS. 7A and 7B depict Nkx2-5 amino acid and nucleotide sequences, respectively.

FIG. 8 depicts Tbx5 amino acid sequences.

FIG. 9 depicts a Tbx5 nucleotide sequence.

FIG. 10 depicts an amino acid sequence alignment of Tbx5 isoform 1 (SEQ ID NO:7), isoform 2 (SEQ ID NO:8), and isoform 3 (SEQ ID NO:9).

### DEFINITIONS

As used herein, the term “stem cell” refers to an undifferentiated cell that can be induced to proliferate. The stem cell is capable of self-maintenance, meaning that with each cell division, one daughter cell will also be a stem cell. Stem cells can be obtained from embryonic, post-natal, juvenile or adult tissue. The term “progenitor cell,” as used herein, refers to an undifferentiated cell derived from a stem cell, and is not itself a stem cell. Some progenitor cells can produce progeny that are capable of differentiating into more than one cell type.

The term “induced pluripotent stem cell” (or “iPS cell”), as used herein, refers to a pluripotent stem cell induced from a somatic cell, e.g., a differentiated somatic cell. iPS cells are capable of self-renewal and differentiation into cell fate-committed stem cells, including neural stem cells, as well as various types of mature cells.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” means the amount of a compound or a number of cells that, when administered to a mammal or other subject for treating a disease, is sufficient to effect such treatment for the disease. The “therapeutically effective amount” will vary depending on the compound or the cell, the disease and its severity and the age, weight, etc., of the subject to be treated.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cardiomyocyte” includes a plurality of such cardiomyocytes and reference to “the myocardial tissue” includes reference to one or more myocardial tissues and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present invention provides methods of generating cardiomyocytes from a cell other than a cardiomyocyte, the methods generally involving contacting the cell with an agent that increases the level and/or activity of a protein that links a transcription factor to a chromatin remodeling complex. The present invention provides a population of cardiomyocytes generated using a subject method; and treatment methods involving introducing the cardiomyocyte population in or around diseased myocardial tissue.

### Methods of Generating a Cardiomyocyte

The present invention provides methods of generating cardiomyocytes from a cell other than a cardiomyocyte, the methods generally involving contacting the cell with an agent that increases the level and/or activity of a protein that links a transcription factor to a chromatin remodeling complex.

In some embodiments, a protein that links a transcription factor to a chromatin remodeling complex is a Brg1-associated factor, e.g., Brg1-associated factor 60c (Baf60c), Baf60a, or Baf60b. Agents that increase the level and/or activity of a Baf60c protein include small molecules, e.g., small molecules that activate the function of a protein such as Baf60c by altering its conformation, by inducing a modification such as phosphorylation, acetylation, or any other change that would promote its activity and/or its interaction with cardiac transcription factors thus leading to activation of cardiac genes. Agents that increase the level and/or activity of a Baf60c protein include nucleic acids. For example, a nucleic acid comprising a nucleotide sequence encoding a Baf60c polypeptide is introduced into a cell, the encoded Baf60c is produced in the cell, and cardiomyogenesis is induced in the cell.

In some embodiments, a subject method comprises contacting a cell other than a cardiomyocyte with: 1) an agent that increase the level and/or activity of a protein that links a transcription factor to a chromatin remodeling complex; and 2) a nucleic acid encoding a cardiac transcription factor. In some embodiments, a subject method comprises introducing into a cell other than a cardiomyocyte one or more nucleic acids comprising nucleotide sequences encoding: 1) a protein that links a transcription factor to a chromatin remodeling complex (e.g., Baf60c); and 2) one or more cardiac transcription factors. Suitable cardiac transcription factors include, but are not limited to, Nkx2-5, Gata4, Tbx5, and Mef2c.

Non-cardiomyocyte cells that are suitable for use in a subject method of inducing cardiomyogenesis include stem cells, progenitor cells, and somatic cells. Suitable cells include, but are not limited to, embryonic stem cells; adult stem cells; induced pluripotent stem (iPS) cells; skin fibroblasts; skin stem cells; cardiac fibroblasts; bone marrow-derived cells; skeletal myoblasts; neural crest cells; and the like. In some embodiments, the stem cell, non-cardiomyocyte somatic cell, or progenitor cell is a human stem cell, a human non-cardiomyocyte somatic cell, or human progenitor cell. In other embodiments, the stem cell, non-cardiomyocyte somatic cell, or progenitor cell is a non-human primate stem cell, a non-human primate non-cardiomyocyte somatic cell, or non-human primate progenitor cell. In other embodiments, the stem cell, non-cardiomyocyte somatic cell, or progenitor cell is a rodent stem cell, a rodent non-cardiomyocyte somatic cell, or a rodent progenitor cell. Stem cells, non-cardiomyocyte somatic cells, and progenitor cells from other mammals (e.g., ungulate cells, e.g., porcine cells) are also contemplated.

In some embodiments, one or more nucleic acids comprising nucleotide sequences encoding 1) a protein that links a transcription factor to a chromatin remodeling complex (e.g., Baf60c); and 2) one or more cardiac transcription factors (e.g., Gata4, Tbx5, Nkx2-5, Mef2c) is introduced into a stem cell, a non-cardiomyocyte somatic cell, or a progenitor cell, generating a genetically modified stem cell, non-cardiomyocyte somatic cell, or progenitor cell. The polypeptides encoded by the introduced nucleic acid(s) are produced in the genetically modified cell; the polypeptides induce cardiomyogenesis in the genetically modified cell. In some embodiments, one or more nucleic acids comprising nucleotide sequences encoding a Baf60c polypeptide, a Gata4 polypeptide, a Tbx5 polypeptide, and an Nkx2-5 polypeptide is introduced into a stem cell, a non-cardiomyocyte somatic cell, or a progenitor cell, generating a genetically modified stem cell, non-cardiomyocyte somatic cell, or progenitor cell. The Baf60, Gat4, Tbx5, and Nkx2-5 polypeptides are produced in the genetically modified stem cell, non-cardiomyocyte somatic cell, or progenitor cell; production of the Baf60, Gat4, Tbx5, and Nkx2-5 polypeptides in the genetically modified cells induces cardiomyogenesis.

In some embodiments, one or more nucleic acids comprising nucleotide sequences encoding 1) a protein that links a transcription factor to a chromatin remodeling complex (e.g., Baf60c); and 2) one or more cardiac transcription factors (e.g., Gata4, Tbx5, Nkx2-5, Mef2c) is introduced into a population of stem cells, a population of non-cardiomyocyte somatic cells, or a population of progenitor cells. A “population of stem cells” includes a population of cells that includes stem cells, which cell population may include cells other than stem cells. Similarly, a “population of non-cardiomyocyte somatic cells” includes a population of non-cardiomyocyte somatic cells, which cell population may include cells other than non-cardiomyocyte somatic cells. A “population of progenitor cells” includes a population of cells that includes progenitor cells, which cell population my include cells other than progenitor cells. In some embodiments, one or more nucleic acids comprising nucleotide sequences encoding 1) a protein that links a transcription factor to a chromatin remodeling complex (e.g., Baf60c); and 2) one or more cardiac transcription factors (e.g., Gata4, Tbx5, Nkx2-5, Mef2c) is introduced into a mixed cell population that includes stem cells and/or non-cardiomyocyte somatic cells and/or progenitor cells.

In some embodiments, the stem cell is a human embryonic stem cell. Human embryonic stem cells that are suitable for use include, but are not limited to, BG01, BG02, and BG03 (provider's code hESBGN-01, hESBGN-02, and hESBGN-03, respectively) (BresaGen, Inc.); SA01 and SA02 (provider's code Sahlgrenska 1 and Sahlgrenska 2, respectively) (Cellartis AB); ES01, ES02, ES03, ES04, ES05, and ES06 (provider's code HES-1, HES-2, HES-3, HES-4, HES-5, and HES-6, respectively) (ES Cell International); TE03, TE04, and TE06 (provider's code 13, 14, and 16, respectively) (National Stem Cell Bank); UC01 and UC06 (provider's code HSF-1 and HSF-6, respectively) (University of California, San Francisco); WA01, WA07, WA09, WA13, and WA17 (provider's code H1, H7, H9, H13, and H14, respectively) (Wisconsin Alumni Research Foundation, WiCell Research Institute). In some embodiments, a human embryonic stem cell has the following characteristics: SSEA-1⁻, SSEA-2⁺, SSEA-3⁺, SSEA-4⁺, TRA 1-60⁺, TRA 1-81⁺, Oct-4⁺, and alkaline phosphatase⁺. Methods of isolating human embryonic cell cells are known in the art. See, e.g., U.S. Pat. No. 7,294,508.

In some embodiments, the stem cell is an induced pluripotent stem (iPS) cell. iPS cells are generated from somatic cells, including skin fibroblasts, using, e.g., known methods. iPS cells produce and express on their cell surface one or more of the following cell surface antigens: SSEA-3, SSEA-4, TRA-1-60, TRA-1-81, TRA-2-49/6E, and Nanog. In some embodiments, iPS cells produce and express on their cell surface SSEA-3, SSEA-4, TRA-1-60, TRA-1-81, TRA-2-49/6E, and Nanog. iPS cells express one or more of the following genes: Oct-3/4, Sox2, Nanog, GDF3, REX1, FGF4, ESG1, DPPA2, DPPA4, and hTERT. In some embodiments, an iPS cell expresses Oct-3/4, Sox2, Nanog, GDF3, REX1, FGF4, ESG1, DPPA2, DPPA4, and hTERT. Methods of generating iPS are known in the art, and any such method can be used to generate iPS. See, e.g., Takahashi and Yamanaka (2006) Cell 126:663-676; Yamanaka et. al. (2007) Nature 448:313-7; Wernig et. al. (2007) Nature 448:318-24; Maherali (2007) Cell Stem Cell 1:55-70.

iPS cells can be generated from somatic cells (e.g., skin fibroblasts) by genetically modifying the somatic cells with one or more expression constructs encoding Oct-3/4 and Sox2. In some embodiments, somatic cells are genetically modified with one or more expression constructs comprising nucleotide sequences encoding Oct-3/4, Sox2, c-myc, and Klf4. In some embodiments, somatic cells are genetically modified with one or more expression constructs comprising nucleotide sequences encoding Oct-4, Sox2, Nanog, and LIN28.

In some embodiments, a subject method comprises: introducing into a non-cardiomyocyte cell (e.g., a stem cell such as an embryonic stem (ES) cell; a somatic cell; or a progenitor cell) one or more nucleic acids comprising nucleotide sequences encoding a Baf60c polypeptide, a Gata4 polypeptide, an Nkx2-5 polypeptide, and a Tbx5 polypeptide, wherein introduction of the one or more nucleic acids results in differentiation of the non-cardiomyocyte (e.g., the ES cell or progenitor cell) into a cardiomyocyte, thereby generating cardiomyocytes. Such cardiomyocytes are useful for, e.g., introducing into an individual who is in need of a cardiomyocyte.

In some embodiments, a subject method comprises: a) inducing a somatic cell from an individual to become a pluripotent stem cell, generating an iPS cell; b) introducing into the iPS cell one or more nucleic acids comprising nucleotide sequences encoding a Baf60c polypeptide, a Gata4 polypeptide, an Nkx2-5 polypeptide, and a Tbx5 polypeptide, wherein introduction of the one or more nucleic acids results in differentiation of the iPS cell into a cardiomyocyte, thereby generating cardiomyocytes. Such cardiomyocytes are useful for, e.g., introducing into the individual from whom the somatic cell was obtained, where the individual is in need of a cardiomyocyte. Alternatively, such cardiomyocytes can be introduced into an individual other than the individual from whom the somatic cell was obtained.

In some embodiments, one or more nucleic acids comprising nucleotide sequences encoding a Baf60c polypeptide, a Gata4 polypeptide, an Nkx2-5 polypeptide, and a Tbx5 polypeptide is introduced into a population of cells that comprises stem cells and/or cardiac progenitor cells; and, as a result, the proportion of cells in the population that are cardiomyocytes or cardiac progenitor cells increases. For example, in some embodiments, introduction of one or more nucleic acids comprising nucleotide sequences encoding a Baf60c polypeptide, a Gata4 polypeptide, an Nkx2-5 polypeptide, and a Tbx5 polypeptide into a cell population that comprises stem cells or cardiac progenitor cells results in differentiation of at least about 10% of the stem cell or progenitor cell population to differentiate into cardiomyocytes. For example, in some embodiments, from about 10% to about 50% of the stem cell or progenitor cell population differentiates into cardiomyocytes. In other embodiments, at least about 50% of the stem cell or progenitor cell population differentiates into cardiomyocytes. For example, in some embodiments, from about 50% to about 60%, from about 60% to about 70%, from about 70% to about 80%, or from about 80% to about 90%, or more, of the stem cell or progenitor cell population differentiates into cardiomyocytes. In some embodiments, introduction of one or more nucleic acids comprising nucleotide sequences encoding a Baf60c polypeptide, a Gata4 polypeptide, an Nkx2-5 polypeptide, and a Tbx5 polypeptide into a stem cell(s) or progenitor cell(s) results in generation of beating cardiac cells from the stem cells or progenitor cells.

A cardiomyocyte will generally express on its cell surface and/or in the cytoplasm one or more cardiac-specific marker. Suitable cardiomyocyte-specific markers include, but are not limited to, cardiac troponin I, cardiac troponin-C, tropomyosin, caveolin-3, GATA-4, myosin heavy chain, myosin light chain-2a, myosin light chain-2v, ryanodine receptor, and atrial natriuretic factor. In some embodiments, introduction of one or more nucleic acids comprising nucleotide sequences encoding a Baf60c polypeptide, a Gata4 polypeptide, an Nkx2-5 polypeptide, and a Tbx5 polypeptide into a stem cell or progenitor cell (e.g., a cardiac progenitor cell) results in generation of a cardiomyocyte that expresses one or more cardiac-specific markers.

In some embodiments, a subject method is carried out in vitro. In other embodiments, a subject method is carried out in vivo. Where a subject method is carried out in vitro, in some embodiments, the cardiomyocytes (or cardiomyocyte precursors) are subsequently introduced into a living organism.

In some embodiments, a subject method is carried out wherein the stem cells, progenitor cells, or somatic cells are present in a matrix. In other embodiments, a subject method involves generating a cardiomyocyte, then associating the cardiomyocyte with a matrix. In these embodiments, e.g., where the method is carried out when cells are present in a matrix, or where a cardiomyocyte generated by a subject method is associated with a matrix, the subject method is suitable for producing an artificial heart tissue.

In some embodiments, a subject method comprises: a) inducing cardiomyogenesis in a population of stem cells or non-cardiomyocyte somatic cells, generating a mixed population of undifferentiated stem cells or non-cardiomyocyte somatic cells and cardiomyocytes; and b) separating cardiomyocytes from the undifferentiated (non-cardiomyocyte) cells. In some embodiments, the separation step comprises contacting the cells with an antibody specific for a cardiomyocyte-specific cell surface marker.

Separation can be carried out using any of a number of well-known methods, including, e.g., any of a variety of sorting methods, e.g., fluorescence activated cell sorting (FACS), negative selection methods, etc. The selected cells are separated from non-selected cells, generating a population of selected (“sorted”) cells. A selected cell population can be at least about 75%, at least about 80%, at least about 85%, at least about 90%, at least about 95%, at least about 98%, at least about 99%, or greater than 99% cardiomyocytes.

Cell sorting (separation) methods are well known in the art. Procedures for separation may include magnetic separation, using antibody-coated magnetic beads, affinity chromatography and “panning” with antibody attached to a solid matrix, e.g. plate, or other convenient technique. Techniques providing accurate separation include fluorescence activated cell sorters, which can have varying degrees of sophistication, such as multiple color channels, low angle and obtuse light scattering detecting channels, impedance channels, etc. Dead cells may be eliminated by selection with dyes associated with dead cells (propidium iodide [PI], LDS). Any technique may be employed which is not unduly detrimental to the viability of the selected cells. Where the selection involves use of one or more antibodies, the antibodies can be conjugated with labels to allow for ease of separation of the particular cell type, e.g. magnetic beads; biotin, which binds with high affinity to avidin or streptavidin; fluorochromes, which can be used with a fluorescence activated cell sorter; haptens; and the like. Multi-color analyses may be employed with the FACS or in a combination of immunomagnetic separation and flow cytometry.

### Genetically Modifying Stem Cells or Progenitor Cells

A subject method of inducing cardiomyogenesis in a stem cell, in a progenitor cell, or in a non-cardiomyocyte somatic cell, or in a population of stem cells, progenitor cells, or non-cardiomyocyte somatic cells, will in some embodiments involve increasing the level of Baf60c in the stem cell(s), progenitor cell(s), or non-cardiomyocyte somatic cell(s). In some embodiments, the method generally involves genetically modifying the stem cell(s), progenitor cell(s), or non-cardiomyocyte somatic cell(s) with an expression construct that comprises a nucleotide sequence encoding Baf60c, wherein the encoded Baf60c is produced in the stem cells, progenitor cells, or non-cardiomyocyte somatic cells, where the Baf60c activates cardiac genes and induces cardiomyogenesis.

A subject method of inducing cardiomyogenesis in a population of stem cells, progenitor cells, or non-cardiomyocyte somatic cells, will in some embodiments involve increasing the level of Baf60c in the stem cells, progenitor cells, or non-cardiomyocyte somatic cells; and genetically modifying the stem cells, progenitor cells, or non-cardiomyocyte somatic cells with an expression construct that comprises a nucleotide sequence encoding one or more cardiac transcription factors. For example, in some embodiments, a subject method involves genetically modifying stem cells, progenitor cells, or non-cardiomyocyte somatic cells with one or more expression constructs comprising nucleotide sequences encoding Baf60c and one or more of Nkx2-5, Gata4, Tbx5, and Mef2c. As another example, in some embodiments, a subject method involves genetically modifying stem cells, progenitor cells, or non-cardiomyocyte somatic cells with one or more expression constructs comprising nucleotide sequences encoding Baf60c, Nkx2-5, Gata4, and Tbx5. Genetic modification can be carried out in vitro or in vivo.

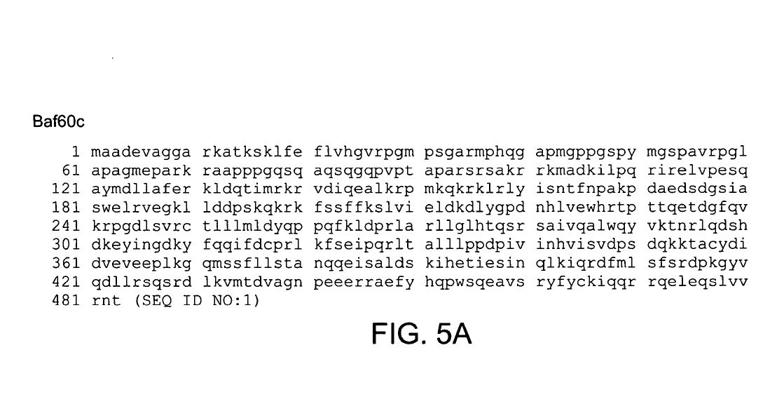

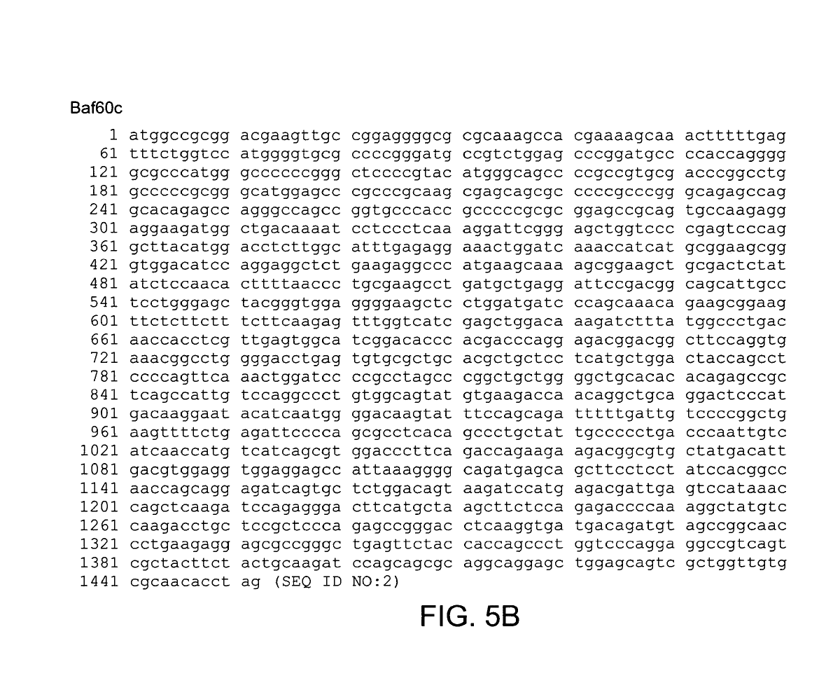

Baf60c is a subunit of an Swi/Snf-like BAF complex, and mediates interactions between cardiac transcription factors (e.g., Gata4, Nkx2-5, Tbx5) and the BAF complex ATPase Brg1. Lickert et al. (2004) Nature 432:107. As used herein, “Baf60c polypeptide” refers to a polypeptide that can link a transcription factor to a chromatin remodeling complex, e.g., can form an association between a transcription factor and a chromatin remodeling complex. For example, a Baf60c polypeptide can form an association between a cardiac transcription factor and a chromatin remodeling complex in a cell (e.g., a non-cardiomyocyte), and induce cardiomyogenesis in the cell. Amino acid sequences of Baf60c polypeptides are known. See, e.g., GenBank Accession No. AAR88511 (Homo sapiens Baf60c isoform 1); GenBank Accession No. AAR88510 (Homo sapiens Baf60c isoform 2); GenBank Accession No. NP_{—}080167 (Mus musculus Baf60c); GenBank Accession No. EDL99343 (Rattus norvegicus Baf60c); and Debril et al (2004) J. Biol. Chem. 279:16677. A Baf60c polypeptide can have a length of from about 450 amino acids to about 470 amino acids, or from about 470 amino acids to about 483 amino acids. The term “Baf60c polypeptide” includes polypeptides having at least about 75%, at least about 80%, at least about 85%, at least about 90%, at least about 95%, at least about 98%, at least about 99%, or 100%, amino acid sequence identity over a contiguous stretch of from about 400 amino acids to about 425 amino acids, from about 425 amino acids to about 450 amino acids, from about 450 amino acids to about 475 amino acids, or from about 475 amino acids to 483 amino acids, of the amino acid sequence depicted in FIG. 5A. In some embodiments, a Baf60c polypeptide lacks the first 13 amino acids of the amino acid sequence depicted in FIG. 5A, or has a methionine residue in place of the first 13 amino acids of the amino acid sequence depicted in FIG. 5A (SEQ ID NO:1). In some embodiments, a Baf60c polypeptide is 470 amino acids in length (e.g., isoform 1). In other embodiments, a Baf60c polypeptide is 483 amino acids in length (e.g., isoform 2).

The term “Baf60c polypeptide” includes fusion polypeptides comprising a Baf60c polypeptide and a non-Baf60c polypeptide (e.g., a “fusion partner” or a “heterologous polypeptide”). Suitable fusion partners include, e.g., epitope tags, proteins that provide a detectable signal; proteins that aid in purification; and the like, as described in more detail below.

A “Baf60c nucleic acid” comprises a nucleotide sequence encoding a Baf60c polypeptide. Nucleotide sequences encoding Baf60c are known in the art. See, e.g., GenBank Accession No. AY450430 (Homo sapiens; encoding Baf60c isoform 2); GenBank Accession No. AY450431 (Homo sapiens; encoding Baf60c isoform 1); GenBank Accession No. NM_{—}025891 (Mus musculus); and GenBank Accession No. CH474020 (Rattus norvegicus). Baf60c nucleic acids suitable for use in a subject method include a nucleic acid comprising a nucleotide sequence having at least about 75%, at least about 80%, at least about 85%, at least about 90%, at least about 95%, at least about 98%, at least about 99%, or 100%, nucleotide sequence identity to a contiguous stretch of from about 1375 nucleotides to about 1400 nucleotides, from about 1400 nucleotides to about 1425 nucleotides, or from about 1425 nucleotides to about 1452 nucleotides, of the nucleotide sequence depicted in FIG. 5B (SEQ ID NO:2).

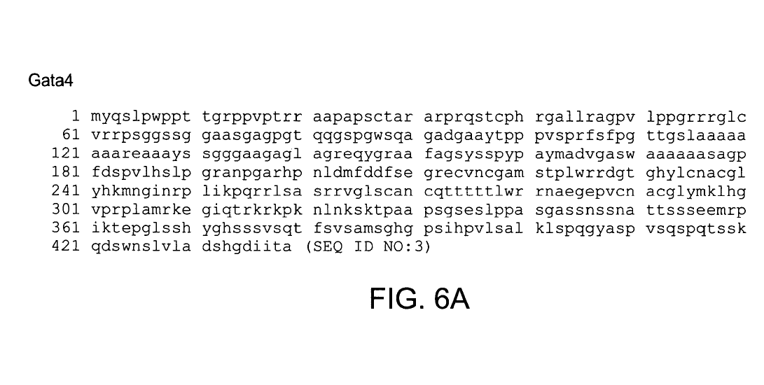

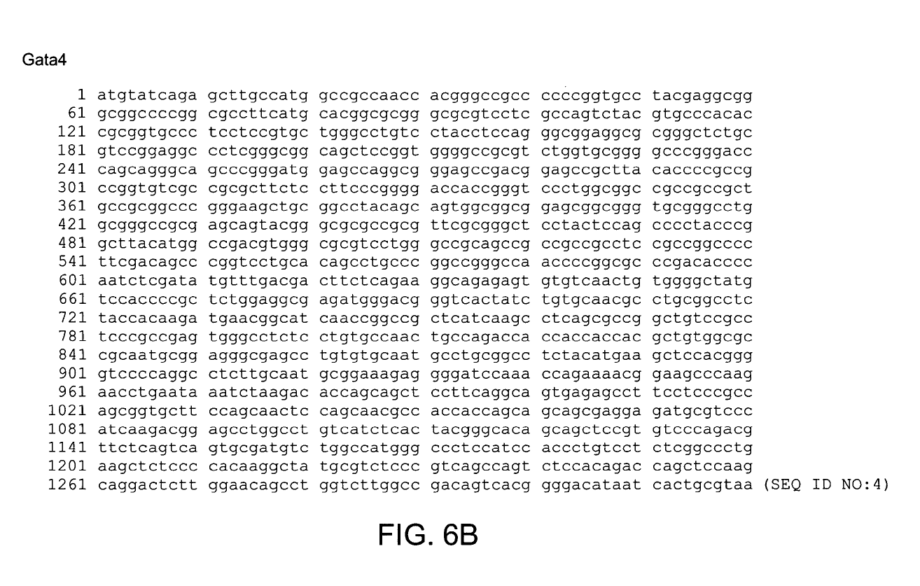

Gata4 is a member of a highly conserved family of proteins that bind identical nucleotide sequences in genomic DNA and regulate expression of similar target genes. Charron et al. (1999) Seminars in cell & developmental biology 10:85-91; Morrisey et al. (1997) J Biol Chem 272:8515-8524; Nemer and Nemer (2003) Dev Biol 254:131-148; Peterkin et al. (2005) Seminars in cell & developmental biology 16:83-94). Gata4 is a cardiac transcription factor that binds nucleotide sequences in certain promoters, including an atrial natriuretic factor promoter, e.g., Gata4 can bind the consensus sequence 5′-a/g-GATA-a/g-3′. Durocher et al. (1997) EMBO J. 16:5687; Small and Krieg (2003) Dev. Biol. 261:116; Watanabe et al. (2000) Proc. Natl. Acad. Sci. USA 97:1624; and Brown et al. (2004) J. Biol. Chem. 279:10659. Amino acid sequences of Gata4 polypeptides are known in the art. See, e.g., GenBank Accession No. BAA11334 (Homo sapiens Gata4); NP_{—}002043 (Homo sapiens Gata4); AAB42015 (Mus musculus Gata4); NP_{—}032118 (Mus musculus Gata4); and Arceci et al. (1993) Mol. Cell Biol. 12:2235. Gata4 polypeptides include a polypeptide having at least about 75%, at least about 80%, at least about 85%, at least about 90%, at least about 95%, at least about 98%, at least about 99%, or 100%, amino acid sequence identity to the amino acid sequence set forth in GenBank Accession No. BAA11334 and depicted in FIG. 6A (SEQ ID NO:3). The term “Gata4 polypeptide” includes polypeptides having at least about 75%, at least about 80%, at least about 85%, at least about 90%, at least about 95%, at least about 98%, at least about 99%, or 100%, amino acid sequence identity over a contiguous stretch of from about 350 amino acids to about 375 amino acids, from about 375 amino acids to about 400 amino acids, or from about 400 amino acids to about 440 (e.g., 439 amino acids) amino acids, of the amino acid sequence depicted in FIG. 6A. A Gata4 polypeptide can have a length of from about 350 amino acids to about 375 amino acids, from about 375 amino acids to about 400 amino acids, from about 400 amino acids to about 425 amino acids, or from about 425 amino acids to about 440 amino acids (e.g., from about 439 amino acids to about 442 amino acids).

The term “Gata4 polypeptide” includes fusion polypeptides comprising a Gata4 polypeptide and a non-Gata4 polypeptide (e.g., a “fusion partner” or a “heterologous polypeptide”). Suitable fusion partners include, e.g., epitope tags, proteins that provide a detectable signal; proteins that aid in purification; and the like, as described in more detail below.

A “Gata4 nucleic acid” comprises a nucleotide sequence encoding a Gata4 polypeptide. Nucleotide sequences encoding Gata4 polypeptides are known in the art. See, e.g., GenBank Accession No. D78260 (encoding a Homo sapiens Gata4); GenBank Accession No. NM_{—}002052; GenBank Accession No. U85046 (encoding a Mus musculus Gata4); and NM_{—}0008092. Gata4 nucleic acids suitable for use in a subject method include a nucleic acid comprising a nucleotide sequence having at least about 75%, at least about 80%, at least about 85%, at least about 90%, at least about 95%, at least about 98%, at least about 99%, or 100%, nucleotide sequence identity to a contiguous stretch of from about 1000 nucleotides to about 1100 nucleotides, from about 1100 nucleotides to about 1200 nucleotides, from about 1200 nucleotides to about 1300 nucleotides, or from about 1300 nucleotides to about 1320 nucleotides, of the nucleotide sequence depicted in FIG. 6B (SEQ ID NO:4).

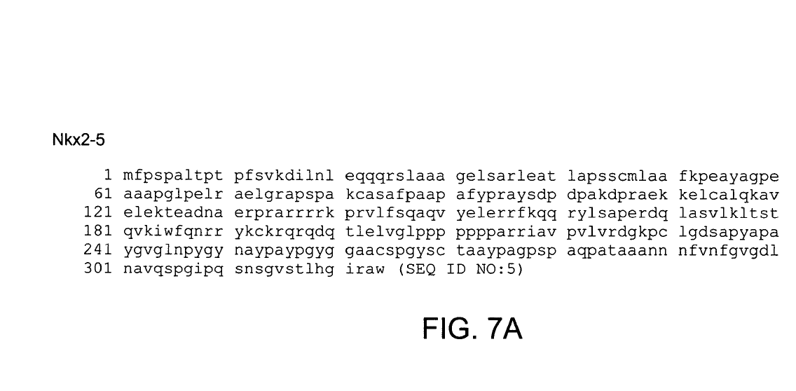

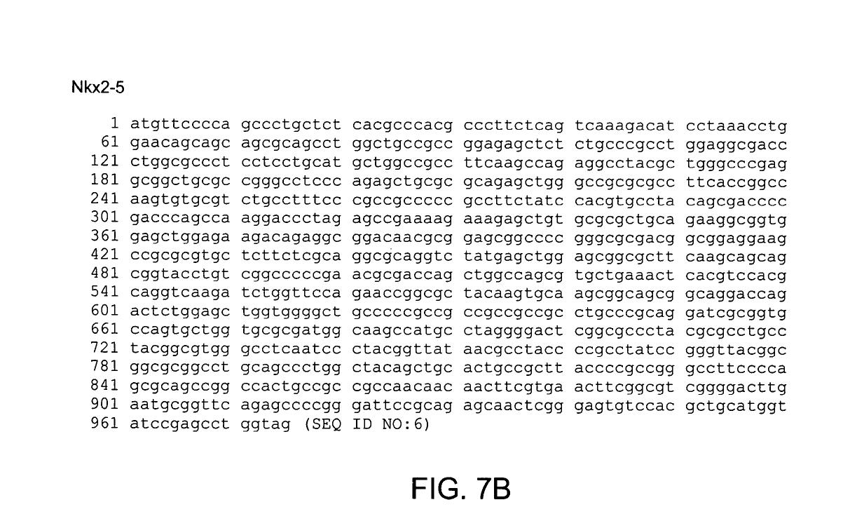

Nkx2-5 is a cardiac transcription factor that binds the atrial natriuretic factor promoter. Durocher et al. (1997) EMBO J. 16:5687. Amino acid sequences of Nkx2-5 polypeptides are known in the art. See, e.g., Turbay et al. (1996) Mol. Med. 2:86; GenBank Accession No. NP_{—}004378 (Homo sapiens Nkx2-5); GenBank Accession No. AAC97934; Mus musculus Nkx2-5); and GenBank Accession No. AAB62696 (Rattus norvegicus Nkx2-5). Nkx2-5 polypeptides include a polypeptide having at least about 75%, at least about 80%, at least about 85%, at least about 90%, at least about 95%, at least about 98%, at least about 99%, or 100%, amino acid sequence identity to the amino acid sequence set forth in GenBank Accession No. NP_{—}004378 and depicted in FIG. 7A (SEQ ID NO:5). The term “Nkx2-5 polypeptide” includes polypeptides having at least about 75%, at least about 80%, at least about 85%, at least about 90%, at least about 95%, at least about 98%, at least about 99%, or 100%, amino acid sequence identity over a contiguous stretch of from about 300 amino acids to about 305 amino acids, from about 305 amino acids to about 310 amino acids, from about 310 amino acids to about 315 amino acids, or from about 315 amino acids to about 324 amino acids, of the amino acid sequence depicted in FIG. 7A. An Nkx2-5 polypeptide can have a length of from about 300 amino acids to about 305 amino acids, from about 305 amino acids to about 310 amino acids, from about 310 amino acids to about 315 amino acids, from about 315 amino acids to about 318 amino acids, or from about 318 amino acids to about 324 amino acids.

The term “Nkx2-5 polypeptide” includes fusion polypeptides comprising a Nkx2-5 polypeptide and a non-Nkx2-5 polypeptide (e.g., a “fusion partner” or a “heterologous polypeptide”). Suitable fusion partners include, e.g., epitope tags, proteins that provide a detectable signal; proteins that aid in purification; and the like, as described in more detail below.

An “Nkx2-5 nucleic acid” comprises a nucleotide sequence encoding an Nkx2-5 polypeptide. Nucleotide sequences encoding Nkx2-5 polypeptides are known in the art. See, e.g., GenBank Accession No. NM_{—}004387 (encoding a Homo sapiens Nkx2-5 polypeptide); GenBank Accession No. AF091351 (encoding a Mus musculus Nkx2-5 polypeptide); and GenBank Accession No. AF006664 (encoding a Rattus norvegicus Nkx2-5 polypeptide). Nkx2-5 nucleic acids suitable for use in a subject method include a nucleic acid comprising a nucleotide sequence having at least about 75%, at least about 80%, at least about 85%, at least about 90%, at least about 95%, at least about 98%, at least about 99%, or 100%, nucleotide sequence identity to a contiguous stretch of from about 900 nucleotides to about 925 nucleotides, from about 925 nucleotides to about 950 nucleotides, or from about 950 nucleotides to about 975 nucleotides, of the nucleotide sequence depicted in FIG. 7B (SEQ ID NO:6).

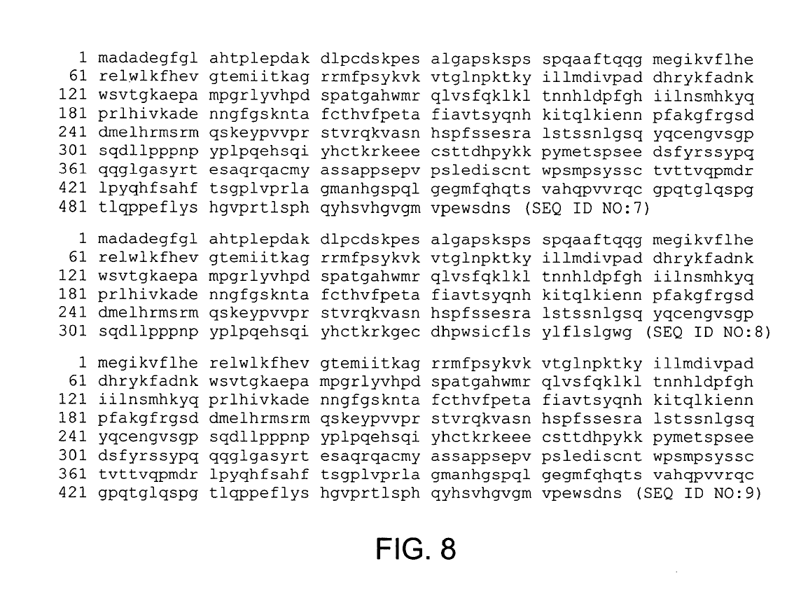

Tbx5 is a transcription factor that binds nucleotide sequences in certain promoters, e.g., Tbx5 can bind the nucleotide sequence 5′-aataTCACACCTgtac-3′ (SEQ ID NO:11. See, e.g., Ghosh et al. (2001) Hum. Mol. Genet. 10:1983; Fan et al. (2003) J. Biol. Chem. 278:8780. Amino acid sequences of Tbx5 polypeptides are known in the art. See, e.g., Wilson and Conlon (2002) Genome Biol. 3:3008.1-3008.7; GenBank Accession Nos. NP_{—}000183, NP_{—}542449; NP_{—}542448; and NP_{—}852259. Nkx2-5 polypeptides include a polypeptide having at least about 75%, at least about 80%, at least about 85%, at least about 90%, at least about 95%, at least about 98%, at least about 99%, or 100%, amino acid sequence identity to any one of the amino acid sequences depicted in FIG. 8 (e.g., any one of SEQ ID NOs:7, 8, and 9). The term “Tbx5 polypeptide” includes polypeptides having at least about 75%, at least about 80%, at least about 85%, at least about 90%, at least about 95%, at least about 98%, at least about 99%, or 100%, amino acid sequence identity over a contiguous stretch of from about 300 amino acids to about 350 amino acids, from about 350 amino acids to about 400 amino acids, from about 400 amino acids to about 450 amino acids, from about 450 amino acids to about 475 amino acids, from about 475 amino acids to about 500 amino acids, or from about 500 amino acids to about 518 amino acids, of one or more of the amino acid sequences depicted in FIG. 8. A Tbx polypeptide can have a length of from about 300 amino acids to about 350 amino acids, from about 350 amino acids to about 400 amino acids, from about 400 amino acids to about 450 amino acids, from about 450 amino acids to about 475 amino acids, from about 475 amino acids to about 500 amino acids, or from about 500 amino acids to about 518 amino acids.

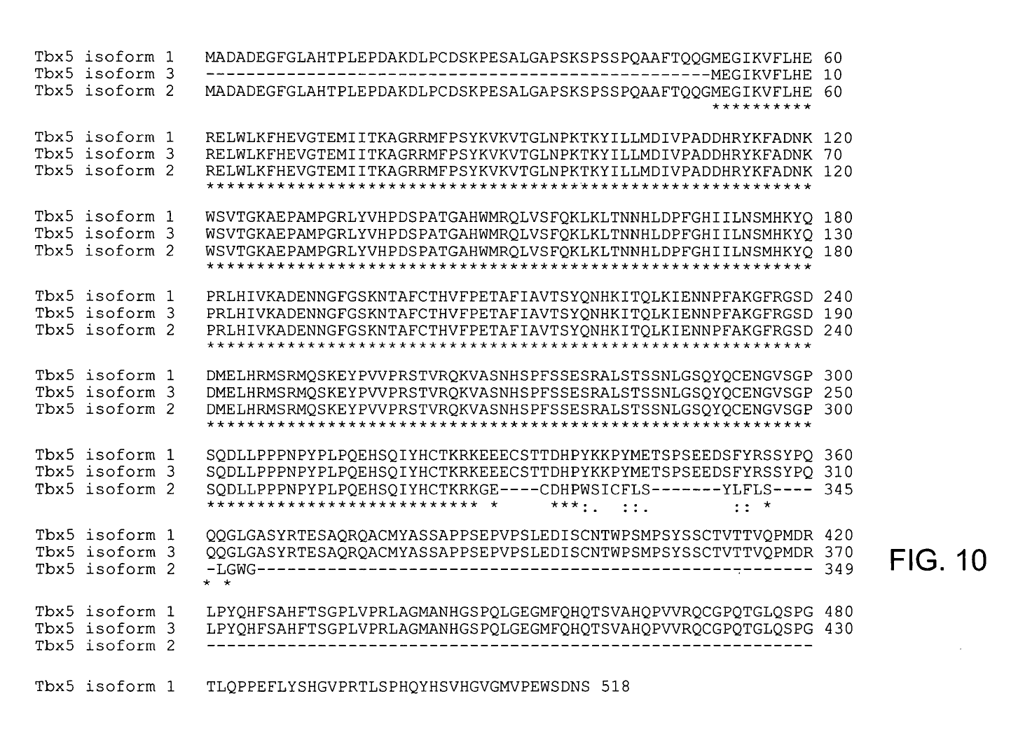

A Tbx polypeptide can be a Tbx5 isoform 1 polypeptide, a Tbx5 isoform 2 polypeptide, or a Tbx isoform 3 polypeptide. An amino acid sequence alignment of Tbx5 isoforms 1, 2, and 3 is depicted in FIG. 10.

The term “Tbx5 polypeptide” includes fusion polypeptides comprising a Tbx5 polypeptide and a non-Tbx5 polypeptide (e.g., a “fusion partner” or a “heterologous polypeptide”). Suitable fusion partners include, e.g., epitope tags, proteins that provide a detectable signal; proteins that aid in purification; and the like, as described in more detail below.

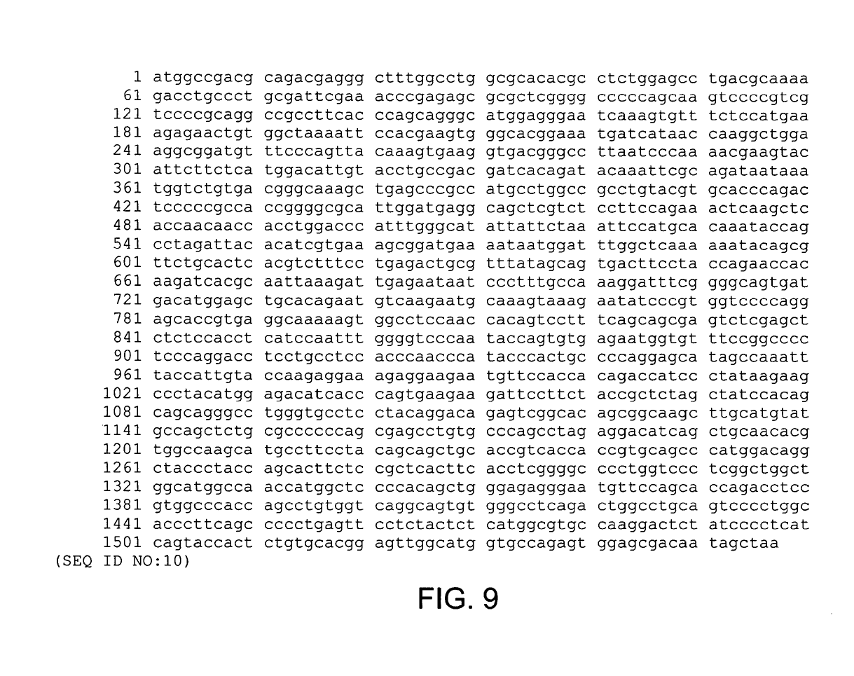

A “Tbx5 nucleic acid” comprises a nucleotide sequence encoding a Tbx5 polypeptide. Nucleotide sequences encoding Tb5 polypeptides are known in the art. See, e.g., GenBank Accession Nos. NM_{—}000192, NM_{—}181486, NM_{—}080717, and NM_{—}080718. Tbx5 nucleic acids suitable for use in a subject method include a nucleic acid comprising a nucleotide sequence having at least about 75%, at least about 80%, at least about 85%, at least about 90%, at least about 95%, at least about 98%, at least about 99%, or 100%, nucleotide sequence identity to a contiguous stretch of from about 1000 nucleotides to about 1100 nucleotides, from about 1100 nucleotides to about 1200 nucleotides, from about 1200 nucleotides to about 1300 nucleotides, from about 1300 nucleotides to about 1400 nucleotides, from about 1400 nucleotides to about 1500 nucleotides, or from about 1500 nucleotides to about 1557 nucleotides, of the nucleotide sequence depicted in FIG. 9 (SEQ ID NO:10).

As noted above, a polypeptide (such as a Baf60c polypeptide, a Gata4 polypeptide, an Nkx2-5 polypeptide, and a Tbx5 polypeptide) can be a fusion polypeptide, comprising a fusion partner. Suitable fusion partners include, but are not limited to, epitope tags; proteins that aid in purification (e.g., (His)ₙ, e.g., 6His; or other metal-binding peptides; glutathione-S-transferase (GST); etc.); and proteins that provide a detectable signal, e.g., fluorescent proteins, enzymes that yield a detectable (e.g., chromogenic, fluorescent, chemiluminescent, etc.) product, and the like. Suitable epitope tags include, e.g., hemagglutinin; a FLAG (flagellin) epitope), c-myc, and the like. Suitable enzymes include, but are not limited to, β-galactosidase, luciferase, horse radish peroxidase, alkaline phosphatase, etc.

Suitable fluorescent proteins include, but are not limited to, green fluorescent protein (GFP; Chalfie, et al., Science 263(5148):802-805 (Feb. 11, 1994); and enhanced GFP (EGFP); Clontech—Genbank Accession Number U55762), blue fluorescent protein (BFP; 1. Quantum Biotechnologies, Inc. 1801 de Maisonneuve Blvd. West, 8th Floor, Montreal (Quebec) Canada H3H 1J9; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; 1. Clontech Laboratories, Inc., 1020 East Meadow Circle, Palo Alto, Calif. 94303), Renilla WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558), a GFP from species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); any of a variety of fluorescent and colored proteins from Anthozoan species, as described in, e.g., Matz et al. (1999) Nature Biotechnol. 17:969-973; U.S. Patent Publication No. 2002/0197676, or U.S. Patent Publication No. 2005/0032085; and the like.

As noted above, a nucleic acid comprising a nucleotide sequence encoding a protein that links a transcription factor to a chromatin remodeling complex and/or a nucleotide sequence encoding a cardiac transcription factor, can be provided in the form of an expression vector. The expression vector can then be introduced into a stem cell, a progenitor cell, or a non-cardiomyocyte somatic cell.

In some embodiments, the expression construct is a viral construct, e.g., a recombinant adeno-associated virus construct (see, e.g., U.S. Pat. No. 7,078,387), a recombinant adenoviral construct, a recombinant lentiviral construct, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:8186, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641648, 1999; Ali et al., Hum Mol Genet 5:591594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

Non-limiting examples of suitable eukaryotic promoters (promoters functional in a eukaryotic cell) include CMV immediate early, HSV thymidine kinase, early and late SV40, long terminal repeats (LTRs) from a retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

In some embodiments, the Baf60c-encoding nucleotide sequence is operably linked to a cardiac-specific transcriptional regulator element (TRE), where TREs include promoters and enhancers. Suitable TREs include, but are not limited to, TREs derived from the following genes: myosin light chain-2, α-myosin heavy chain, AE3, cardiac troponin C, and cardiac actin. Franz et al. (1997) Cardiovasc. Res. 35:560-566; Robbins et al. (1995) Ann. N.Y. Acad. Sci. 752:492-505; Linn et al. (1995) Circ. Res. 76:584-591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051. In some embodiments, the nucleotide sequences encoding one or more of Gata4, Nkx2-5, and Tbx5 are operably linked to a cardiac-specific TRE, as described above for Baf60c. For example, in some embodiments, the nucleotide sequences encoding Gata4, Nkx2-5, and Tbx5 are all operably linked to a cardiac-specific TRE.

### Cardiomyocyte Compositions

The present invention provides cardiomyocyte compositions generated using a subject method. In some embodiments, the cardiomyocyte composition is artificial heart tissue.

In some embodiments, a cardiomyocyte is present in a liquid medium together with one or more components. Suitable components include, but are not limited to, salts; buffers; stabilizers; protease-inhibiting agents; cell membrane- and/or cell wall-preserving compounds, e.g., glycerol, dimethylsulfoxide, etc.; nutritional media appropriate to the cell; and the like.

### Artificial Heart Tissue

The artificial heart tissue can be used for allogenic or autologous transplantation into an individual in need thereof. To produce artificial heart tissue, a matrix can be provided which is brought into contact with the stem cells or progenitor cells, where the stem cells or progenitor cells are induced to undergo cardiomyogenesis using a subject method, as described above. This means that this matrix is transferred into a suitable vessel and a layer of the cell-containing culture medium is placed on top (before or during the differentiation of the expanded stem cells or progenitor cells). Alternatively, a cardiomyocyte generated using a subject method can be associated with a matrix after the cardiomyocyte is generated.

The term “matrix” should be understood in this connection to mean any suitable carrier material to which the cells are able to attach themselves or adhere in order to form the corresponding cell composite, i.e. the artificial tissue. In some embodiments, the matrix or carrier material, respectively, is present already in a three-dimensional form desired for later application. For example, bovine pericardial tissue is used as matrix which is crosslinked with collagen, decellularized and photofixed.

For example, a matrix (also referred to as a “biocompatible substrate”) is a material that is suitable for implantation into a subject onto which a cell population can be deposited. A biocompatible substrate does not cause toxic or injurious effects once implanted in the subject. In one embodiment, the biocompatible substrate is a polymer with a surface that can be shaped into the desired structure that requires repairing or replacing. The polymer can also be shaped into a part of a structure that requires repairing or replacing. The biocompatible substrate provides the supportive framework that allows cells to attach to it, and grow on it. Cultured populations of cells can then be grown on the biocompatible substrate, which provides the appropriate interstitial distances required for cell-cell interaction.

### Utility

A subject method for generating cardiomyocytes is useful for generating cardiomyocytes. Cardiomyocytes, and compositions (e.g., tissues) comprising such cardiomyocytes, generated using a subject method, can be used in various research applications, treatment methods, and screening methods.

### Research Applications

A subject method can be used to generate cardiomyocytes or cardiac progenitors for research applications. Research applications include, e.g., introduction of the cardiomyocytes or cardiac progenitors into a non-human animal model of a disease (e.g., a cardiac disease) to determine efficacy of the cardiomyocytes or cardiac progenitors in the treatment of the disease; use of the cardiomyocytes in screening methods to identify candidate agents suitable for use in treating cardiac disorders; and the like. For example, a cardiomyocyte or cardiac progenitor generated using a subject method can be contacted with a test agent, and the effect, if any, of the test agent on a biological activity of the cardiomyocyte or cardiac progenitor can be assessed, where a test agent that has an effect on a biological activity of the cardiomyocyte or cardiac progenitor is a candidate agent for treating a cardiac disorder. As another example, a cardiomyocyte or cardiac progenitor generated using a subject method can be introduced into a non-human animal model of a cardiac disorder, and the effect of the cardiomyocyte or cardiac progenitor on ameliorating the disorder can be tested in the non-human animal model.

### Screening Methods

As noted above, a cardiomyocyte generated using a subject method can be used in a screening method to identify candidate agents for treating a cardiac disorder. For example, a cardiomyocyte generated using a subject method is contacted with a test agent; and the effect, if any, of the test agent on a parameter associated with normal or abnormal cardiomyocyte function is determined. Alternatively, artificial heart tissue generated by a subject method can be contacted with a test agent; and the effect, if any, of the test agent on a parameter associated with normal or abnormal cardiomyocyte function is determined. Such parameters include, but are not limited to, beating; expression of a cardiomyocyte-specific marker; electric signals associated with heart beating; and the like.

### Treatment Methods

A subject method is useful for generating cardiomyocytes, which can be introduced into an individual in need thereof, e.g., a cardiomyocyte generated using a subject method can be introduced on or adjacent to existing heart tissue in an individual. A subject method is useful for replacing damaged heart tissue (e.g., ischemic heart tissue). A subject method is useful for stimulating endogenous stem cells resident in the heart to undergo cardiomyogenesis. Where a subject method involves introducing (implanting) a cardiomyocyte into an individual, allogenic or autologous transplantation can be carried out.

The present invention provides methods of treating a cardiac disorder in an individual, the method generally involving administering to an individual in need thereof a therapeutically effective amount of: a) a population of cardiomyocytes prepared using a subject method; b) a population of cardiac progenitors prepared using a subject method; and c) an artificial heart tissue prepared using a subject method. For example, in some embodiments, a subject method comprises: i) inducing a stem cell to differentiate into a cardiomyocyte; and ii) introducing the cardiomyocyte into an individual in need thereof. In other embodiments, a subject method comprises: i) inducing a stem cell or progenitor cell to differentiate into a cardiomyocyte (e.g., by introducing into the stem cell or progenitor cell one or more nucleic acids comprising nucleotide sequences encoding a Baf60c polypeptide, a Gata4 polypeptide, an Nkx2-5 polypeptide, and a Tbx5 polypeptide); and ii) introducing the cardiomyocyte into an individual in need thereof. In other embodiments, a subject method comprises: i) generating artificial heart tissue by: a) inducing a stem cell or progenitor cell to differentiate into a cardiomyocyte (e.g., by introducing into the stem cell or progenitor cell one or more nucleic acids comprising nucleotide sequences encoding a Baf60c polypeptide, a Gata4 polypeptide, an Nkx2-5 polypeptide, and a Tbx5 polypeptide); and b) associating the cardiomyocyte with a matrix, to form artificial heart tissue; and ii) introducing the artificial heart tissue into an individual in need thereof. In other embodiments, a subject method comprises: i) generating an iPS cell from a somatic cell from an individual; ii) inducing the iPS cell to differentiate into a cardiomyocyte (e.g., by introducing into the iPS cell one or more nucleic acids comprising nucleotide sequences encoding a Baf60c polypeptide, a Gata4 polypeptide, an Nkx2-5 polypeptide, and a Tbx5 polypeptide); and iii) introducing the cardiomyocyte into the individual from whom the somatic cell was obtained, which individual is in need of a cardiomyocyte. In some embodiments, a subject method comprises: i) generating an iPS cell from a somatic cell from an individual; ii) inducing the iPS cell to differentiate into a cardiomyocyte (e.g., by introducing into the iPS cell one or more nucleic acids comprising nucleotide sequences encoding a Baf60c polypeptide, a Gata4 polypeptide, an Nkx2-5 polypeptide, and a Tbx5 polypeptide); iii) associating the cardiomyocyte with a matrix, to generate artificial heart tissue; and iv) introducing the artificial heart tissue into the individual from whom the somatic cell was obtained, which individual is in need of the artificial heart tissue.

In other embodiments, a subject method comprises: i) generating an iPS cell from a somatic cell from an individual (e.g., a donor); ii) inducing the iPS cell to differentiate into a cardiomyocyte (e.g., by introducing into the iPS cell one or more nucleic acids comprising nucleotide sequences encoding a Baf60c polypeptide, a Gata4 polypeptide, an Nkx2-5 polypeptide, and a Tbx5 polypeptide); and iii) introducing the cardiomyocyte into a recipient individual other than the donor individual from whom the somatic cell was obtained, which recipient individual is in need of a cardiomyocyte. In some embodiments, a subject method comprises: i) generating an iPS cell from a somatic cell from an individual (e.g., a donor); ii) inducing the iPS cell to differentiate into a cardiomyocyte (e.g., by introducing into the iPS cell one or more nucleic acids comprising nucleotide sequences encoding a Baf60c polypeptide, a Gata4 polypeptide, an Nkx2-5 polypeptide, and a Tbx5 polypeptide); iii) associating the cardiomyocyte with a matrix, to generate artificial heart tissue; and iv) introducing the artificial heart tissue into a recipient individual other than the donor individual from whom the somatic cell was obtained, which recipient individual is in need of the artificial heart tissue.

A subject method is useful for generating artificial heart tissue, e.g., for implanting into a mammalian subject. A subject method is useful for replacing damaged heart tissue (e.g., ischemic heart tissue). A subject method is useful for stimulating endogenous stem cells or non-cardiomyocyte somatic cells resident in the heart to undergo cardiomyogenesis. Where a subject method involves introducing (implanting) a cardiomyocyte into an individual, allogenic or autologous transplantation can be carried out.

Individuals in need of treatment using a subject method include, but are not limited to, individuals having a congenital heart defect; individuals suffering from a condition that results in ischemic heart tissue, e.g., individuals with coronary artery disease; and the like. A subject method is useful to treat degenerative muscle disease, e.g., familial cardiomyopathy, dilated cardiomyopathy, hypertrophic cardiomyopathy, restrictive cardiomyopathy, or coronary artery disease with resultant ischemic cardiomyopathy.

For administration to a mammalian host, a cardiomyocyte population generated using a subject method can be formulated as a pharmaceutical composition. A pharmaceutical composition can be a sterile aqueous or non-aqueous solution, suspension or emulsion, which additionally comprises a physiologically acceptable carrier (i.e., a non-toxic material that does not interfere with the activity of the active ingredient). Any suitable carrier known to those of ordinary skill in the art may be employed in a subject pharmaceutical composition. The selection of a carrier will depend, in part, on the nature of the substance (i.e., cells or chemical compounds) being administered. Representative carriers include physiological saline solutions, gelatin, water, alcohols, natural or synthetic oils, saccharide solutions, glycols, injectable organic esters such as ethyl oleate or a combination of such materials. Optionally, a pharmaceutical composition may additionally contain preservatives and/or other additives such as, for example, antimicrobial agents, anti-oxidants, chelating agents and/or inert gases, and/or other active ingredients.

In some embodiments, a cardiomyocyte population is encapsulated, according to known encapsulation technologies, including microencapsulation (see, e.g., U.S. Pat. Nos. 4,352,883; 4,353,888; and 5,084,350). Where the cardiomyocytes are encapsulated, in some embodiments the cardiomyocytes are encapsulated by macroencapsulation, as described in U.S. Pat. Nos. 5,284,761; 5,158,881; 4,976,859; 4,968,733; 5,800,828 and published PCT patent application WO 95/05452.

In some embodiments, a cardiomyocyte population is present in a matrix, as described below.

A unit dosage form of a cardiomyocyte population can contain from about 10³ cells to about 10⁹ cells, e.g., from about 10³ cells to about 10⁴ cells, from about 10⁴ cells to about 10⁵ cells, from about 10⁵ cells to about 10⁶ cells, from about 10⁶ cells to about 10⁷ cells, from about 10⁷ cells to about 10⁸ cells, or from about 10⁸ cells to about 10⁹ cells.

A cardiomyocyte population can be cryopreserved according to routine procedures. For example, cryopreservation can be carried out on from about one to ten million cells in “freeze” medium which can include a suitable proliferation medium, 10% BSA and 7.5% dimethylsulfoxide. Cells are centrifuged. Growth medium is aspirated and replaced with freeze medium. Cells are resuspended as spheres. Cells are slowly frozen, by, e.g., placing in a container at −80° C. Cells are thawed by swirling in a 37° C. bath, resuspended in fresh proliferation medium, and grown as described above.

### Artificial Heart Tissue

In some embodiments, a subject method comprises: a) inducing cardiomyogenesis in a population of stem cells, progenitor cells, or non-cardiomyocyte somatic cells in vitro, e.g., where the stem cells, progenitor cells, or non-cardiomyocyte somatic cells are present in a matrix, wherein a population of cardiomyocytes (or cardiomyocyte precursors) is generated; and b) implanting the population of cardiomyocytes into or on an existing heart tissue in an individual. Thus, the present invention provides a method for generating artificial heart tissue in vitro; and implanting the artificial heart tissue in vivo.

The artificial heart tissue can be used for allogenic or autologous transplantation into an individual in need thereof. To produce artificial heart tissue, a matrix can be provided which is brought into contact with the stem cells or progenitor cells, where the stem cells or progenitor cells are induced to undergo cardiomyogenesis using a subject method, as described above. This means that this matrix is transferred into a suitable vessel and a layer of the cell-containing culture medium is placed on top (before or during the differentiation of the expanded stem cells or progenitor cells). The term “matrix” should be understood in this connection to mean any suitable carrier material to which the cells are able to attach themselves or adhere in order to form the corresponding cell composite, i.e. the artificial tissue. In some embodiments, the matrix or carrier material, respectively, is present already in a three-dimensional form desired for later application. For example, bovine pericardial tissue is used as matrix which is crosslinked with collagen, decellularized and photofixed.

For example, a matrix (also referred to as a “biocompatible substrate”) is a material that is suitable for implantation into a subject onto which a cell population can be deposited. A biocompatible substrate does not cause toxic or injurious effects once implanted in the subject. In one embodiment, the biocompatible substrate is a polymer with a surface that can be shaped into the desired structure that requires repairing or replacing. The polymer can also be shaped into a part of a structure that requires repairing or replacing. The biocompatible substrate provides the supportive framework that allows cells to attach to it, and grow on it. Cultured populations of cells can then be grown on the biocompatible substrate, which provides the appropriate interstitial distances required for cell-cell interaction.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

Baf60c, a cardiac-enriched subunit of the polymorphic Swi/Snf-like BAF complexes, was recently identified as an important modulator of cardiac gene expression and morphogenesis (Lickert et al. (2004), supra). Mouse embryos with reduced levels of Baf60c have complex heart morphogenesis defects and impaired activation of several cardiac genes. Here it is shown that, while the cardiac transcription factors Tbx5, Gata4 and Nkx2-5 are ineffective at inducing cardiogenesis alone, inclusion of Baf60c results in induction of ectopic contractile heart tissue in cultured mouse embryos. The minimal requirement for induction of cardiac genes consists of Baf60c and a single DNA-binding factor, Gata4. Thus, when paired with Baf60c, GATA factors appear to be key for the initiation of cardiogenesis. Furthermore, it is demonstrated here that mouse embryos lacking both Gata4 and Gata6, which are functionally redundant, do not initiate cardiac myocyte differentiation, resulting in acardia. The transcriptional basis of cardiogenesis was defined as the combination of GATA factors linked to the recruitment of the BAF chromatin-remodelling complex via Baf60c. This provides a robust mechanism for the precise control of cellular differentiation via two layers of tissue-specificity: one at the DNA-binding level and the other in the form of cell type-specific chromatin remodelling complexes.

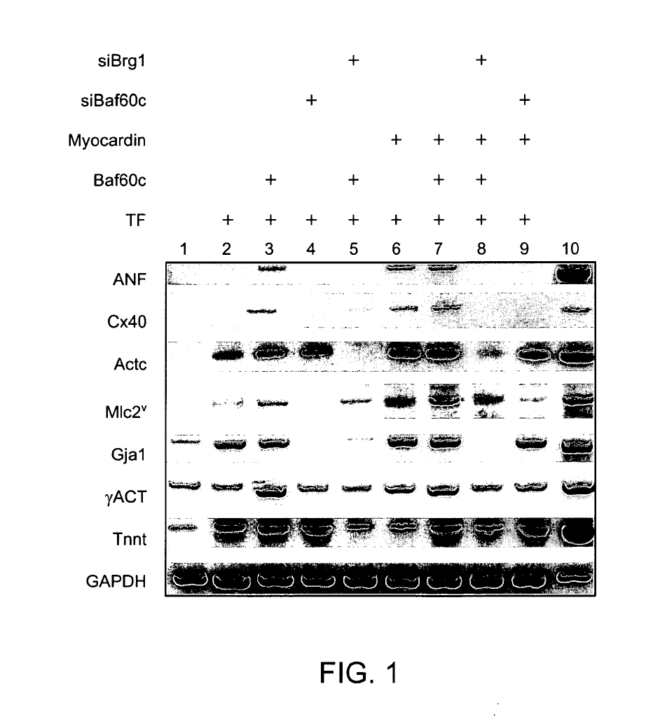

FIG. 1 depicts Baf60c-mediated activation of cardiac genes in 10T1/2 fibroblast cells. Reverse-transcriptase-mediated polymerase chain reaction (RT-PCR) shows expression of cardiac and other genes. 10T1/2 cells were transfected (+sign indicates inclusion in transfection), and RT-PCR was performed 40 hours later. Lane 1 is untransfected cells, while lane 10 is embryonic heart. TF: cardiac transcription factors (Nkx2-5, Tbx5, Gata4); siBrg1: RNA interference to deplete the BAF complex ATPase Brg1; siBaf60c: RNA interference to deplete endogenous Baf60c. Note that the cardiac genes ANF and Cx40 are activated by TF+Baf60c (lane 3); myocardin+TF (lane 6), and myocardin+TF+Baf60c; the lane 6 treatment is ineffectual when Baf60c is depleted (lane 9), indicating that this combination relies on the recruitment of BAF chromatin remodeling complexes via Baf60c. Expression of other cardiac genes (Actc, Mlc2v) is activated by TF, and enhanced by inclusion of Baf60c. gACT and Tnnt are not cardiac specific, but are cardiac-expressed genes, and their expression levels are increased by TF+Baf60c (Lane 3).

### Example 2

### Induction of Cardiogenesis

### Materials and Methods

### Mouse Embryology

Mouse embryo transfections and culture were carried out according to a published technique (Takeuchi et al. (2007) Proc Natl Acad Sci USA 104:846-851; Yamamoto et al. (2004) Nature 428:387-392) with modifications. The modifications were mainly the timing and location of the transfection: at E7.0, embryos were injected posteriorly under the visceral endoderm.

### Generation of Gata4−/− Gata6−/− ES Cell Lines, Embryoid Bodies and Embryos

The production of Gata4^{-/-} and Gata6^{-/-} ES cells have been described (Watt et al. (2004) Proc Natl Acad Sci USA 101:12573-12578; Zhao et al. (2005) Mol Cell Biol 25:2622-2631). Gata4^{-/-}; Gata6^{-/-} ES cells were generated as follows. First, a targeting vector, pGATA4loxPDT, was produced, that contains a Neo-tk cassette flanked by two loxP sites that was inserted into the SmaI site 85 bp upstream of Gata4 exon 3 (Watt et al. (2004), supra). In addition, a single loxP site was inserted into the BamHI site between exons 5 and 6. Cre-mediated recombination between the two outermost loxP sites deletes both zinc fingers domains and the transactivation domain of GATA4, resulting in complete loss of function and the absence of detectable protein (Watt et al. (2004), supra). R1 ES cells were targeted with the pGATA4loxPDT vector, and homologous recombination was detected by the production of a unique 5 kb Sad fragment due to the addition of a SacI site within Neo-tk and a unique 2-kb EcoRI fragment due to the addition of an EcoRI site in loxPc. Gata4+/loxPneo ES cells were electroporated with a Cre expression plasmid and selected in 2 μM gancyclovir; deletion of the Neo-tk cassette could occurred by recombination between loxPa and loxPb, deleting only the Neo-tk cassette and leaving behind a single loxPa/b site and the loxPc site flanking exons 3-5. This produces the Gata4 loxP allele with an 11-kb Sad fragment and a 2-kb EcoRI fragment. Alternatively, recombination between loxP a and loxP c deletes the neo-tk cassette and 2-kb of genomic DNA, leaving behind a single loxP a/c site. This produces a Gata4—allele with a 9-kb SacI fragment and a 2-kb EcoRI fragment. Gata4−/− ES cells were generated by targeting Gata4+/−ES cells with same targeting vector, followed by transient expression of Cre as described above. The genotype of Gata4−/− ES cells was confirmed by Southern blot. The Gata6 gene was targeted in Gata4−/− ES cells using a published targeting vector (Morrisey et al. (1998) Genes Dev 12:3579-3590; Zhao et al. (2005) supra). This vector contains a Pgk-Neo cassette, which replaces exons encoding both zinc fingers and results in a Gata6 null allele. Gata4−/− ES cells were electroporated with the targeting vector, collected colonies that were resistant to growth in G418 (350 μg/ml), and identified Gata6+/− ES cells by genomic Southern blot analyses. Gata4−/−Gata6+/− ES cells were cultured in culture medium supplemented with an elevated concentration (1.5 mg/ml) of G418, as described (Zhao et al. (2005) supra), and identified Gata4−/−Gata6−/− ES cells by Southern blot analyses.

EBs were produced after treating ES cells with Noggin and withdrawing leukemia inhibitory factor, following a published procedure (Yuasa et al., 2005). In three independent experiments, EBs were collected at day 9 after removal of leukemia inhibitory factor. Embryos were produced directly from ES cells by tetraploid embryo complementation as described elsewhere (Nagy et al. (2003) Manipulating the Mouse Embryo. A Laboratory Manual, 3rd edn (Cold Spring Harbor, N.Y., Cold Spring Harbor Laboratory Press); Nagy et al. (1993) Proc Natl Acad Sci USA 90:8424-8428); 12 noon on the day a vaginal plug in surrogate mothers was identified was considered as E0.5.

Oligonucleotide Arrays, RT-PCR and Real-Time qRT-PCR

Total RNA was collected from three independent control and Gata4−/−Gata6−/− EB preparations. Probes were prepared by Affymetrix protocols. Each sample was hybridized to an individual Affymetrix GeneChip Mouse Genome 2.0 array, and data were analyzed in R/Bioconductor with the Affy/affyPLM package, and RMA (robust multi-array average) intensity in log2 scale was generated for each probe set (gene) as described (Bolstad et al. (2003) Bioinformatics (Oxford, England) 19:185-193; Irizarry et al. (2003) Biostatistics (Oxford, England) 4:249-264). Linear models were fitted for each gene on the sample group to derive estimated mutant effects and their associated significance, using the limma package in R/Bioconductor. Moderated t-statistics of the two-sample tests and the associated p-values were calculated, as well as B-statistics (logOdds), the log posterior odds ratio that a gene is differentially expressed or not. The cutoff was arbitrarily chosen as FDR<0.05 and fold change >4×. Semi-quantitative RT-PCR was carried out as described (Watt et al. (2004) supra; Zhao et al. (2005) supra). Real time quantitative RT-PCR was performed using SYBR green incorporation with reactions run on a BioRad iCycler, following the manufacturer's protocol with empirically optimized primer pairs. All oligonucleotide sequences are available on request, with the exception of proprietary oligonucleotides purchased from Superarray Bioscience Corp that were optimized for qRT-PCR amplification of Mesp1 (cat#PPM24667A) or Mesp2 (cat#PPM27883A).

### Immunohistochemistry, Antibodies, Histochemistry and in Situ Hybridization

Embryos were collected, then fixed with 4% paraformaldehyde and stored in 70% ethanol. Embryos were processed for paraffin sections or cryosections. Immunohistochemistry was performed on either whole embryos or sections after microwave antigen retrieval as discussed elsewhere. The following primary antibodies were used: anti-SMA (Sigma A-2547; 1:800); MF20, anti-myosin heavy chain (Developmental Hybridoma Bank; 1:1000), FoxA1 (C-20, Santa Cruz, sc-6553; 1:400), sarcomeric actin (Sigma A-2172; 1:800). Whole mount in situ hybridization was performed using digoxigenin-labeled probes generated by in vitro transcription (Roche) and standard procedures.

### Results

### Transcriptional Induction of Cardiogenesis In Vivo Requires Baf60c

In vivo transient transfections were performed in cultured mouse embryos (Takeuchi et al. (2007) supra; Yamamoto et al. (2004), supra) with combinations of three transcription factors that are important for activation of several cardiac genes (Olson (2006) Science 313:1922-1927; Srivastava (2006) Cell 126:1037-1048): the T-box transcription factor Tbx5, the homeodomain transcription factor Nkx2-5, and the zinc-finger transcription factor Gata4, with or without Baf60c. After transfection, assays for expression of the early marker of cardiac differentiation, Actc, were carried out. Control transfections (EGFP) or Tbx5/Nkx2-5/Gata4 did not result in induction of Actc. Cotransfection of Tbx5/Nkx2-5/Gata4+Baf60c, however, led to markedly expanded and ectopic activation of Actc. Inclusion of Myocardin (Mycd), a transcription factor that activates some cardiac genes de novo (Creemers et al. (2006) Mol Cell 23:83-96), did not potentiate this effect; Mycd transfection by itself resulted in scarce Actc-positive cells in a few (4/13) embryos, but no beating tissue (0/4), and the combination of Tbx5Nkx2-5/Gata4/Mycd did not result in ectopic cardiac differentiation at all (0/6).

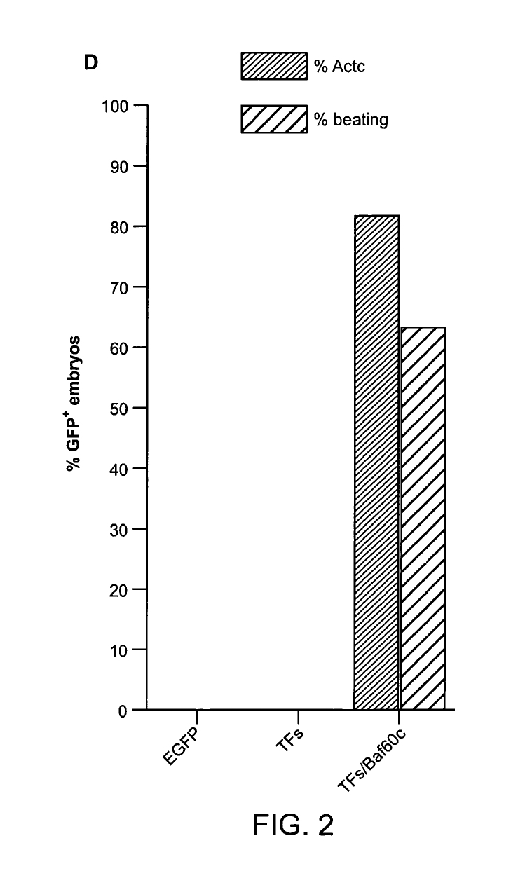

In embryos co-transfected with transcription factors and Baf60c, ectopic Myl2 mRNA and a-tropomyosin protein, which are additional specific markers of the embryonic heart, were detected. Induction of cardiogenesis was limited to the mesoderm and was largely confined to transfected cells, strongly suggesting a cell-autonomous effect. Strikingly, as shown in FIG. 2, ectopic beating tissue was observed in normally non-cardiogenic mesoderm transfected with Tbx5/Nkx2-5/Gata4+Baf60c (9/16 embryos), indicative of a full cardiac program being induced. Ectopic contractile tissue was achieved even though the endogenous cardiac field was not yet beating, indicating accelerated cardiac differentiation. Thus, it was demonstrated that a simple combination of transcription factors and Baf60c can induce complete cardiac myocyte differentiation in vivo.

### Gata Factors and Baf60c are Sufficient for Aspects of Cardiogenesis

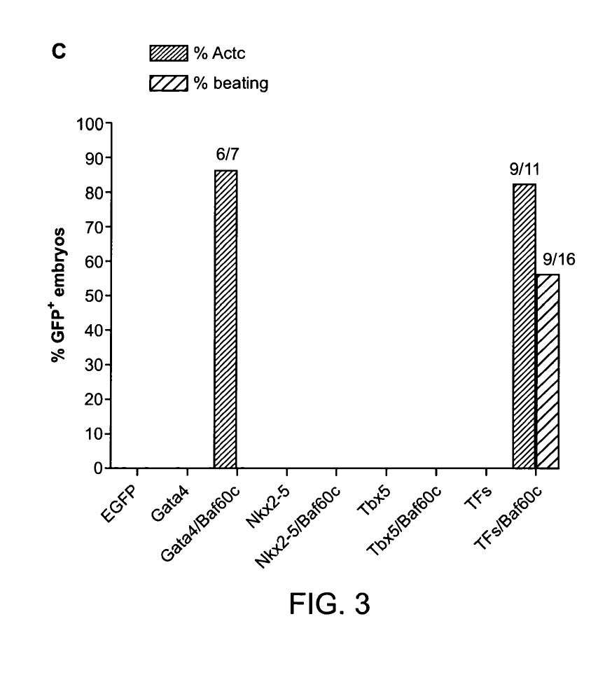

Experiments were carried out to define the minimal set of factors required for cardiogenic induction in vivo. As shown in FIG. 3, Gata4+Baf60c efficiently induced ectopic Actc (6/7 embryos); however, neither Nkx2-5+Baf60c nor Tbx5+Baf60c could achieve this. Nkx2-5, but not Tbx5, was also induced by Gata4+Baf60c, indicating that the additional activity of Nkx2-5 helps to establish the cardiogenic program initiated by Gata4+Baf60c. Although Gata4+Baf60c were sufficient to initiate expression of characteristic cardiac myocyte genes, they did not induce contractile tissue alone. It was hypothesized that the lack of beating might be due to the absence of spontaneous depolarization. Indeed, the pacemaker channel gene Hcn4 (Stieber et al. (2003) Proc Natl Acad Sci USA 100:15235-15240) was induced in embryos transfected with Tbx5/Nkx2-5/Gata4+Baf60c, but not in embryos transfected with Gata4+Baf60c. Thus, even though the onset of cardiac differentiation can be induced by Gata factors with Baf60c, additional input from Tbx5 is necessary for full cardiac cell maturation.

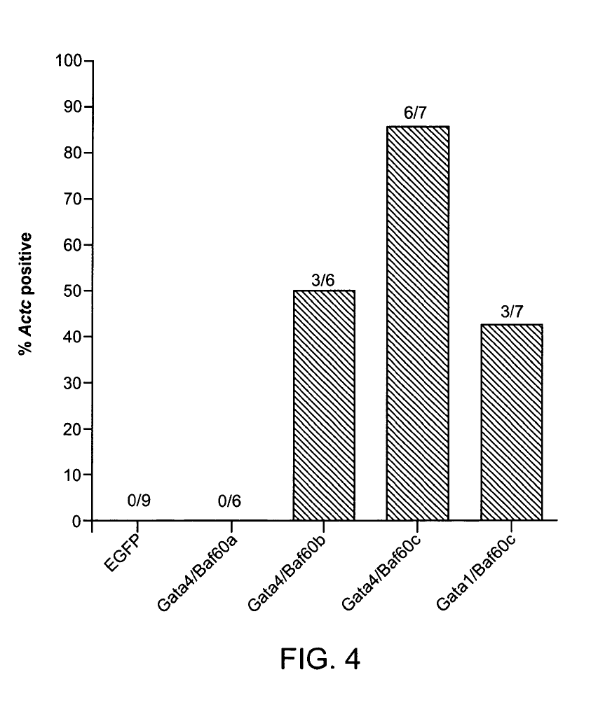

Finally, experiments were carried out to determine if Gata4 and Baf60c have specific cardiogenic properties, or if related factors not normally expressed in the heart could perform the same function. To do so, Gata4 was replaced by the haematopoietic cell transcription factor Gata1 (Whitelaw et al. (1990) Mol Cell Biol 10:6596-6606 and replaced Baf60c by Baf60a or Baf60b, which are not expressed in the heart (Lickert et al. (2004) supra). As shown in FIG. 4, Gata1 could replace Gata4, but with reduced efficiency, and Baf60b (but not Baf60a) could replace Baf60c, again with reduced potency. These results indicate a degree of specificity conferred by intrinsic properties of Gata4 and Baf60c.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of generating a cardiomyocyte, the method comprising introducing into a stem cell, a non-cardiomyocyte somatic cell, or a progenitor cell one or more nucleic acids comprising a nucleotide sequence encoding a protein that links a transcription factor to a chromatin remodeling complex, and a nucleotide sequence encoding a cardiac transcription factor, wherein said introducing provides for differentiation of the stem cell, non-cardiomyocyte somatic cell, or progenitor cell into a cardiomyocyte.

2. The method of claim 1, wherein the protein that links a transcription factor to a chromatin remodeling complex is selected from Baf60c, Baf60a, and Baf60b.

3. The method of claim 2, wherein the protein comprises an amino acid sequence having at least about 75% amino acid sequence identity to the amino acid sequence set forth in SEQ ID NO:1.

4. The method of claim 1, wherein the stem cell is selected from an embryonic stem cell or a progeny thereof, an adult stem cell, an induced pluripotent stem cell.

5. The method of claim 4, wherein the adult stem cell is a skin stem cell or a bone marrow-derived stem cell.

6. The method of claim 1, wherein the non-cardiomyocyte somatic cell is selected from a skin fibroblast, a cardiac fibroblast, a skeletal myoblast, and a neural crest cell.

7. The method of claim 1, wherein the cell is a human cell.

8. The method of claim 1, wherein the cardiac transcription factor is selected from Nkx2-5, Gata4, Tbx5, and Mef2c.

9. The method of claim 1, wherein the cardiac transcription factors are a Gata4 polypeptide, a Nkx2-5 polypeptide, and a Tbx5 polypeptide.

10. The method of claim 9, wherein the Gata4 polypeptide comprises an amino acid sequence having at least 75% amino acid sequence identity to the amino acid sequence set forth in SEQ ID NO:3.

11. The method of claim 9, wherein the Nkx2-5 polypeptide comprises an amino acid sequence having at least 75% amino acid sequence identity to the amino acid sequence set forth in SEQ ID NO:5.

12. The method of claim 9, wherein the Tbx5 polypeptide comprises an amino acid sequence having at least 75% amino acid sequence identity to the amino acid sequence set forth in any one of SEQ ID NOs:7, 8, and 9.

13. The method of claim 1, wherein the cardiomyocyte is associated with a matrix.

14. A method of generating a cardiomyocyte, the method comprising:
a) generating an induced pluripotent cell from a somatic cell; and
b) introducing into the iPS cell one or more nucleic acids comprising a nucleotide sequence encoding a protein that links a transcription factor to a chromatin remodeling complex, and a nucleotide sequence encoding a cardiac transcription factor.

15. A method of generating artificial heart tissue, the method comprising:
a) introducing into a stem cell, a non-cardiomyocyte somatic cell, or a progenitor cell one or more nucleic acids comprising a nucleotide sequence encoding a protein that links a transcription factor to a chromatin remodeling complex, and a nucleotide sequence encoding a cardiac transcription factor, wherein said introducing provides for differentiation of the stem cell, the non-cardiomyocyte, or the progenitor cell into a cardiomyocyte;
b) associating the cardiomyocyte with a matrix, thereby generating artificial heart tissue.

16. A method of treating diseased myocardium, the method comprising:
a) introducing into a population of stem cells, non-cardiomyocyte somatic cells, or progenitor cells one or more nucleic acids comprising a nucleotide sequence encoding a protein that links a transcription factor to a chromatin remodeling complex, and a nucleotide sequence encoding a cardiac transcription factor, wherein said introducing provides for differentiation of stem cells, non-cardiomyocytes, or progenitor cells in the stem cell, non-cardiomyocyte, or progenitor cell population into cardiomyocytes, generating a cardiomyocyte population; and
b) implanting the cardiomyocyte population into diseased myocardial tissue in vivo.

17. The method of claim 16, wherein the stem cell is selected from an embryonic stem cell or a progeny thereof, an adult stem cell, an induced pluripotent stem cell, and a somatic cell.

18. A cardiomyocyte generated by the method of claim 1.

19. A cardiomyocyte comprising one or more exogenous nucleic acids comprising a nucleotide sequence encoding a protein that links a transcription factor to a chromatin remodeling complex, and a nucleotide sequence encoding a cardiac transcription factor.

20. A composition comprising artificial heart tissue generated by the method of claim 15.
